SEQUENCE LISTING (6 sequences)

SEQ ID NO: 1 - DNA, 35 bp, Artificial Sequence
  Single stranded oligonucleotide primer
gctctagaggatccttgttttccgcagcaacagat

SEQ ID NO: 2 - DNA, 30 bp, Artificial Sequence
  Single stranded oligonucleotide primer
cgggatccaagcttacctggtggccgaggc

SEQ ID NO: 3 - DNA, 35 bp, Artificial Sequence
  Single stranded oligonucleotide primer
gctacgtaggtctcgcatgctctaccaattgcatg

SEQ ID NO: 4 - DNA, 30 bp, Artificial Sequence; identical to SEQ ID NO: 2
  Single stranded oligonucleotide primer

SEQ ID NO: 5 - DNA, 25 bp, Artificial Sequence
  Single stranded oligonucleotide primer
gcaagcttcgactgcacggtgcacc

SEQ ID NO: 6 - DNA, 30 bp, Artificial Sequence; identical to SEQ ID NO: 2
  Single stranded oligonucleotide primer

(19) United States
(12) Patent Application Publication
(10) Pub. No.: US 2003/0143703 A1
(43) Pub. Date: Jul. 31, 2003

(54) Recombinant microorganism expressing polyhydroxyalkanoate biosynthesis enzyme and intracellular PHA depolymerase

(76) Inventors: Sang-Yup Lee, Taejon (KR); Young Lee, Taejon (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/299,025

(22) Filed: Nov. 15, 2002

Related U.S. Application Data
(63) Continuation of application No. PCT/KR00/00787, filed on Jul. 20, 2000.

(30) Foreign Application Priority Data
May 16, 2000 (KR) 2000/0026158

(51) Int. Cl. C12P 7/62; C08G 63/87; C08G 63/82; C08G 63/78; C12N 1/21; C12N 15/74

(52) U.S. Cl. 435/135; 435/252.3; 435/471; 435/320.1; 528/274

(57) ABSTRACT

The present invention provides recombinant plasmids containing a gene coding for polyhydroxyalkanoate (PHA) biosynthesis enzyme and a gene coding for intracellular PHA depolymerase, and a process for preparing (R)-hydroxycarboxylic acid employing the same. In accordance with the present invention, optically pure (R)-hydroxycarboxylic acid can be prepared by culturing E. coli transformed with a recombinant plasmid which expresses intracellular PHA depolymerase of Ralstonia eutropha and PHA biosynthesis enzyme of Alcaligenes latus to give (R)-hydroxycarboxylic acid. The process for preparing (R)-hydroxycarboxylic acid of the invention can be successfully employed in large-scale continuous process with a high productivity by carrying out PHA synthesis and degradation in a simultaneous manner, while enjoying the benefits of simple procedures for harvesting cells and disposing the cell wastes.

### BACKGROUND OF THE INVENTION

1. Field of the Invention

The present invention relates to recombinant plasmids containing a gene coding for polyhydroxyalkanoate (PHA) biosynthesis enzyme and a gene coding for intracellular PHA depolymerase, and a process for preparing (R)-hydroxycarboxylic acids employing the same, more specifically, to recombinant plasmids containing a gene coding for polyhydroxyalkanoate (PHA) biosynthesis enzyme and a gene coding for intracellular PHA depolymerase in cis, and a process for preparing optically pure (R)-hydroxycarboxylic acids by introducing the said plasmids into E. coli and culturing the recombinant microorganisms wherein biosynthesis and depolymerization of PHA occur simultaneously.

2. Description of the Prior Art

Since (R)-hydroxycarboxylic acid carrying two functional groups, i.e., hydroxyl group (—OH) and carboxyl group (—COOH) can provide chiral center easily in organic syntheses of a variety of useful materials and also the two functional groups can be converted into other forms easily, it can be widely used as a chiral precursor compound in fine chemical fields. It can be used as an intermediate for synthesis of antibiotics, vitamins, aromatics and pheromones, and applied for the development of nonpeptide ligands which can be used in the designs of medical and pharmaceutical products, and used as a precursor of novel pharmaceuticals, especially, carbapenem antibiotics which draw attentions as a substitute of penicillin (see: Lee et al., Biotechnol. Bioeng., 65:363-368, 1999). As an example, the process for synthesis of (+)-thiennamycin from methyl-(R)-3-hydroxybutyrate has been reported (see: Chiba and Nakai, Chem. Lett., 651-654, 1985).

Polyhydroxyalkanoates (PHAs) formed by ester linkage of hydroxycarboxylic acids are a class of polyesters that are synthesized and accumulated in many species of microorganisms as storage materials for energy and carbon. Since the monomer, hydroxycarboxylic acid, comprising PHA have only (R)-type optical activity due to the optical specificity of biosynthesis enzyme except a few cases such as 4-hydroxybutyric acid of which optical isomers do not exist, optically pure (R)-3-hydroxycarboxylic acids can be produced simply by depolymerizing biosynthesized PHA. Processes for preparing (R)-3-hydroxybutyrate, alkyl-(R)-3-hydroxybutyrate, or alkyl-(R)-3-hydroxyvalerate via chemical degradation of poly-(hydroxybutyrate) (PHB) or poly (3-hydroxybutyrate-co-3-hydroxyvalerate) (PHB/V) have been reported (see: Seebach et al., Org. Synth., 71:39-47, 1992; Seebach and Zuger, Helvetica Chim. Acta, 65:495-503, 1982). However, the process for preparing (R)-3-hydroxybutyrate by chemical method has intrinsic disadvantages that the yield is lowered by complicated steps comprising culture of microorganism, recovery of cells, isolation of polymers, followed by depolymerization and isolation/purification, and requirement of a large quantity of organic solvents. Moreover, a great deal of microbial cell mass are produced as a byproduct, limiting trial of producing (R)-hydrocarboxylic acids merely to (R)-3-hydroxybutyrate and (R)-3-hydroxyvalerate.

Recently, the present inventors have reported the process for preparing various (R)-3-hydroxycarboxylic acids including (R)-3-hydroxybutyrate via autodegradation (depolymerization) process employing PHA depolymerase which occurs naturally with PHA biosynthesis enzyme system in the PHA-producing microorganisms (see: Lee et al., Biotechnol. Bioeng., 65:363-368, 1999). The autodegradation method is more efficient than conventional chemical methods, for example, after overproducing PHB in Alcaligenes latus by fermentation, incubation for 30 min under a proper pH condition would allow the microorganism to degrade PHB into (R)-hydroxybutyrate with over 95% optical purity which is then released into a medium (see: Lee et al., Biotechnol. Bioeng., 65:363-368, 1999). The autodegradation method applied to produce various (R)-3-hydroxycarboxylic acids follows batch processes in which PHA is accumulated and then degraded. If biosynthesis and degradation of PHA could occur simultaneously in a continuous process, it could be expected that the yield of hydoxycarboxylic acids from the substrate is increased. In view of above situation, there is a continuing need to develop the process for preparing (R)-3-hydroxycarboxylic acids by a simple continuous process to produce (R)-3-hydroxycarboxylic acids in a more efficient and economical way.

The general mechanism of biosynthesis and degradation of PHA in the microorganism is as follows. When a microorganism is under unbalanced growth condition of sufficient carbon source and limited essential elements such as nitrogen, phosphate, or magnesium, the enzymes in PHA biosynthesis pathway are expressed, and PHA is synthesized and accumulated inside cells using excessive carbon source (see: Lee, Biotechnol. Bioeng., 49:1-14, 1996). Later, when supply of essential elements are resumed, PHA is degraded into its monomer, (R)-3-hydroxycarboxylic acid, by the action of PHA depolymerase and oligomer hydrolysis enzymes (see: Muller and Seebach, Angew. Chem. Int. Ed. Engl., 32:477-502, 1993).

The mechanism of recycling of (R)-3-hydroxycarboxylic acid in the metabolic pathway of microorganism has been established only for (R)-3-hydroxybutyrate as follows. By the action of (R)-3-hydroxybutyrate dehydrogenase, (R)-3-hydroxybutyrate produced is converted into acetoacetate which is recycled in the metabolic pathway of the microorganism (see: Muller and Seebach, Angew. Chem. Int. Ed. Engl., 32:477-502, 1993; Lee et al., Biotechnol. Bioeng., 65:363-368, 1999). Therefore, both PHA biosynthesis enzyme system and PHA depolymerase are required for the production of (R)-3-hydroxycarboxylic acids in microorganisms, and for the production of (R)-3-hydroxybutyrate, it is preferable to inhibit or remove (R)-3-hydroxybutyrate dehydrogenase activity.

The present inventors and many other researchers have conducted researches on the development of efficient process for PHA production employing recombinant E. coli, and in case of PHB or PHB/V, it could be accumulated up to 80% of total dried cell mass (see: Slater et al., J. Bacteriol., 170:4431-4436, 1988; Schubert et al., 170, 5837-5847, 1988; Kim et al., Biotechnol. Lett., 14:811-816, 1992; Fidler and Dennis, FEMS Microbiol. Rev., 103:231-236, 1992; Lee et al., J. Biotechnol., 32:203-211, 1994; Lee et al., Ann. NY Acad. Sci., 721:43-53, 1994; Lee et al., Biotechnol. Bioeng., 44:1337-1347, 1994; Lee and Chang, J. Environ. Polymer Degrad., 2:169-176, 1994; Lee and Chang, Can. J. Microbiol., 41:207-215, 1995; Yim et al., Biotechnol. Bioeng., 49:495-503, 1996; Lee and Lee, J. Environ. Polymer Degrad., 4:131-134, 1996; Wang and Lee, Appl. Environ. Microbiol., 63:4765-4769, 1997; Wang and Lee, Biotechnol. Bioeng., 58:325-328, 1998; Lee, Bioprocess Eng., 18:397-399, 1998; Choi et al., Appl. Environ. Microbiol, 64:4897-4903, 1998; Wong and Lee, Appl. Microbial. Biotechnol., 50:30-33, 1998; and, Lee et al., Int. J. Biol. Macromol., 25:31-36, 1999).

Meanwhile, E. coli in nature neither synthesize PHA as an intracellular storage material for energy, nor have PHA depolymerase. Moreover, it is considered that E. coli do not have (R)-3-hydroxybutyrate dehydrogenase which converts (R)-3-hydroxybutyrate into acetoacetate. PHA-synthesizing recombinant E. coli constructed by introducing genes coding for PHA synthesis-relating enzymes from other species neither degrade PHA synthesized and accumulated in the cells because the recombinant E. coli carry PHA biosynthesis enzyme system only (see: Lee, Trends Biotechnol., 14:98-105, 1996; Lee, Nature Biotechnol., 15:17-18, 1997). Therefore, the present inventors have perceived that (R)-3-hydroxycarboxylic acid, especially, (R)-3-hydroxybutyrate can be produced efficiently by cointroducing/coexpressing genes for PHA-synthesizing enzyme and PHA depolymerase in recombinant E. coli, furthermore, (R)-3-hydroxybutyrate would not be metabolized to acetoacetate in the absence of (R)-3-hydroxybutyrate dehydrogenase.

### SUMMARY OF THE INVENTION

The present inventors have made an effort to prepare optically active (R)-3-hydroxycarboxylic acids by cointroducing/coexpressing genes for PHA-synthesizing enzyme and PHA depolymerase in recombinant E. coli, thus, they constructed recombinant plasmids containing a gene for intracellular PHA depolymerase of Ralstonia eutropha along with a gene for PHA biosynthesis enzyme of Alcaligenes latus or Ralstonia eutropha, and have found that E. coli transformed with the said plasmids can secret hydroxycarboxylic acids including (R)-3-hydroxybutyrate and (R)-3-hydroxyvalerate into the medium.

A primary object of the present invention is, therefore, to provide recombinant plasmids containing a gene coding for polyhydroxyalkanoate (PHA) biosynthesis enzyme and a gene coding for intracellular PHA depolymerase in cis.

The other object of the invention is to provide microorganisms transformed with the said recombinant plasmids.

Another object of the invention is to provide a process for preparing (R)-3-hydroxycarboxylic acids by culturing the said transformed microorganisms.

### BRIEF DESCRIPTION OF THE DRAWINGS

The above and the other objects and features of the present invention will become apparent from the following descriptions given in conjunction with the accompanying drawings, in which:

FIG. 1 is a genetic map of the recombinant plasmid of the invention, pJC4Red.

FIG. 2 is a genetic map of the recombinant plasmid of the invention, pSYL105Red.

FIG. 3 is a genetic map of the recombinant plasmid of the invention, pSYL107Red.

FIG. 4 is a genetic map of the recombinant plasmid of the invention, pJC4Red-trc.

FIG. 5 is a genetic map of the recombinant plasmid of the invention, pSYL105Red-trc.

FIG. 6 is a genetic map of the recombinant plasmid of the invention, pSYL107Red-trc.

### DETAILED DESCRIPTION OF THE INVENTION

The process for preparing (R)-hydroxycarboxylic acid of the invention comprises steps of culturing E. coli transformed with recombinant plasmids expressing intracellular PHA depolymerase of Ralstonia eutropha and PHA biosynthesis enzyme of Ralstonia eutropha or Alcaligenes latus in a simultaneous manner, and isolating (R)-hydrocarboxylic acids from the culture.

In accordance with the present invention, (R)-3-hydroxybutyrate and its dimer can be obtained in secreted form in the medium by culturing E. coli transformed with recombinant plasmids expressing PHA biosynthesis enzyme and PHA depolymerase. The secreted (R)-3-hydroxybutyrate and its dimer can be fractionated employing LC or HPLC under a specified condition. Dimer can be degraded into (R)-3-hydroxybutyrate by heating under an alkaline condition (see: Lee et al., Biotechnol. Bioeng., 65:363-368, 1999), if necessary.

A gene for intracellular PHA depolymerase of Ralstonia eutropha carrying an intrinsic constitutive promoter was obtained by PCR (polymerase chain reaction) of chromosomal DNA of Ralstonia eutropha using the nucleotide sequences registered in GenBank™ and then cloned into a plasmid pSYL105 (see: Lee et al., Biotechnol. Bioeng., 44:1337-1347, 1994) containing a gene for PHA biosynthesis enzyme of Ralstonia eutropha, a plasmid pSYL107 (see: Lee, Biotechnol. Lett., 16:1247-1252, 1994; Korean Patent No. 164282) containing a gene for PHA biosynthesis enzyme and a gene ftsZ which is related to the cell division, in cis, and a plasmid pJC4 (KCTC 0481BP) (see: Choi et al., Appl. Environ. Microbiol., 64:4897-4903, 1998) containing a gene for PHA biosynthesis enzyme of Alcaligenes latus, respectively, to construct three recombinant plasmids, pSYL105Red, pSYL107Red and pJC4Red. And, additional three plasmids, pSYL105Red-trc, pSYL107Red-trc and pJC4Red-trc were constructed by replacing the intrinsic constitutive promoter of the above-cloned gene for PHA depolymerase of Ralstonia eutropha with inducible trc promoter.

Six recombinant E. coli containing a gene for PHA biosynthesis enzyme and a gene for PHA depolymerase were obtained by transforming E. coli XL1-Blue (Stratagene Cloning System, U.S.A.) with six plasmids constructed above by employing elecroporation technique, and cultured in the medium containing an appropriate carbon source to obtain (R)-3-hydroxycarboxylic acids, of which concentrations were measured.

From the above results, it was clearly demonstrated that (R)-3-hydroxycarboxylic acids can be efficiently prepared by culturing a recombinant E. coli into which a gene for intracellular PHA depolymerase of Ralstonia eutropha has been introduced with a gene for PHA biosynthesis enzyme of Alcaligenes latus or Ralstonia eutropha, thus, optimal culture conditions were established.

The culture conditions determined in this way to prepare (R)-3-hydroxybutyrate were desirably 30 to 70 hours of culture time for the recombinant E. coli cotransformed with a gene for PHA depolymerase of Ralstonia eutropha and a gene for PHA biosynthesis enzyme of Alcaligenes latus or Ralstonia eutropha; for the recombinant E. coli cotransformed with a gene for PHA depolymerase of Ralstonia eutropha inducible by trc promoter and a gene for PHA biosynthesis enzyme of Alcaligenes latus or Ralstonia eutropha, culture time prior to induction was desirably 24 to 72 hours, and extended culture time after induction was desirably 2 to 8 hours.

The culture conditions to prepare (R)-3-hydroxybutyrate/(R)-3-hydroxyvalerate were desirably 15 to 70 hours of culture time for the recombinant E. coli cotransformed with a gene for PHA depolymerase of Ralstonia eutropha and a gene for PHA biosynthesis enzyme of Alcaligenes latus or Ralstonia eutropha; for the recombinant E. coli cotransformed with a gene for PHA depolymerase of Ralstonia eutropha inducible by trc promoter and a gene for PHA biosynthesis enzyme of Alcaligenes latus or Ralstonia eutropha, culture time prior to induction was desirably 10 to 72 hours, and extended culture time after induction was desirably 2 to 8 hours.

The present invention is further illustrated in the following examples, which should not be taken to limit the scope of the invention.

### EXAMPLE 1

### Cloning of a Gene for Intracellular PHA Depolymerase of Ralstonia eutropha

In order to clone a gene coding for intracellular PHA depolymerase of Ralstonia eutropha into E. coli, the gene isolated from Ralstonia eutropha by the method of Marmur (see: Marmur, J. Mol. Biol., 3:208-218, 1961) was amplified by PCR with primer 1, 5′-GCTCTAGAGGATCCTTGTTTTCCGCAGCAACAGAT-3′ (SEQ ID NO: 1) and primer 2, 5′-CGGGATCCAAGCTTACCTGGTGGCCGAGGC-3′ (SEQ ID NO: 2) which were prepared from nucleotide sequence (see: Saito and Saegusa, GenBank Sequence Database, AB017612, 1999) of the gene for intracellular PHA depolymerase of Ralstonia eutropha. PCR was performed under a following condition: one cycle of denaturation at 95° C. for 5 min; 30 cycles of denaturation at 95° C. for 50 sec, annealing at 55° C. for 1 min and 10 sec, and extension at 72° C. for 3 min; plus one cycle of extension at 72° C. for 7 min. DNA obtained by PCR was digested with BamHI and then subjected to agarose gel electrophoresis to isolate approximately 1.4 kbp DNA fragment which was subsequently ligated into BamHI site of pUC19 plasmid (see: Sambrook et al., Molecular Cloning, A Laboratory Manual 2nd Ed., Cold Spring Harbor Laboratory Press, Cold Spring Harbor, N.Y., 1989) to give a recombinant plasmid, pUC19Red. And, E. coli XL1-Blue was transformed with the recombinant plasmid, pUC19Red by electroporation technique and transformants were selected on LB agar plate (yeast extract, 5 g/L; trypton, 10 g/L; NaCl, 10 g/L, bacto-agar, 15 g/L) containing ampicillin (50 μg/l) to obtain a recombinant E. coli, XL1-Blue/pUC19Red. The cloned DNA fragment was subjected to analysis of nucleotide sequence, which was then compared to nucleotide sequences registered in GenBank™, to confirm that the DNA fragment contains a gene for PHA depolymerase of Ralstonia eutropha including intrinsic constitutive promoter region. The said plasmid, pUC19Red, was digested with HindIII and then subjected to agarose gel electrophoresis to isolate DNA fragment of 1.4 kbp containing intracellular PHA depolymerase gene of Ralstonia eutropha. The isolated DNA fragment was cloned into a plasmid pJC4 (see: Choi et al., Appl. Environ. Microbiol., 64:4897-4903, 1998) which contains a gene for PHA biosynthesis enzyme of Alcaligenes latus and two different plasmids pSYL105 and pSYL107 (see: Lee, Biotechnol. Lett., 15:1247-1252, 1994; Wang and Lee, Appl. Environ. Microbiol., 63:4765-4769, 1997) which contain a gene for PHA biosynthesis enzyme of Ralstonia eutropha, respectively, to construct recombinant plasmids, pJC4Red, pSYL105Red and pSYL107Red.

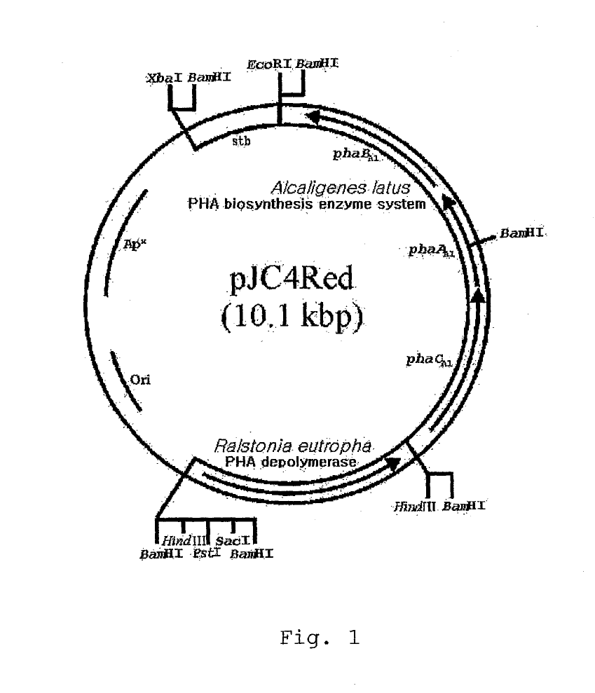

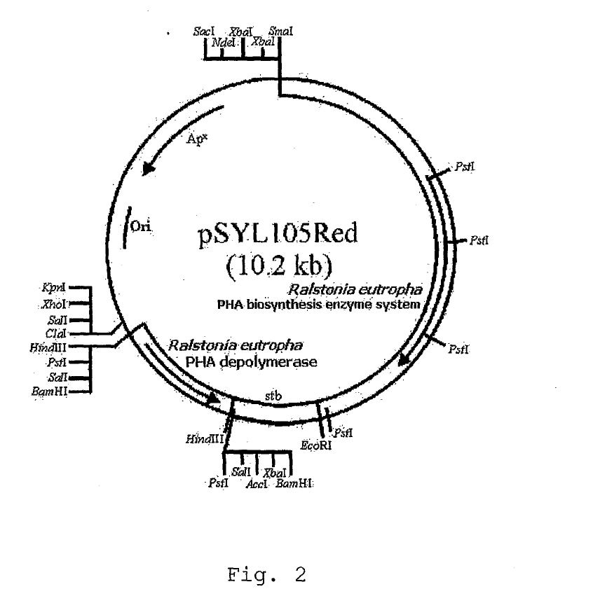

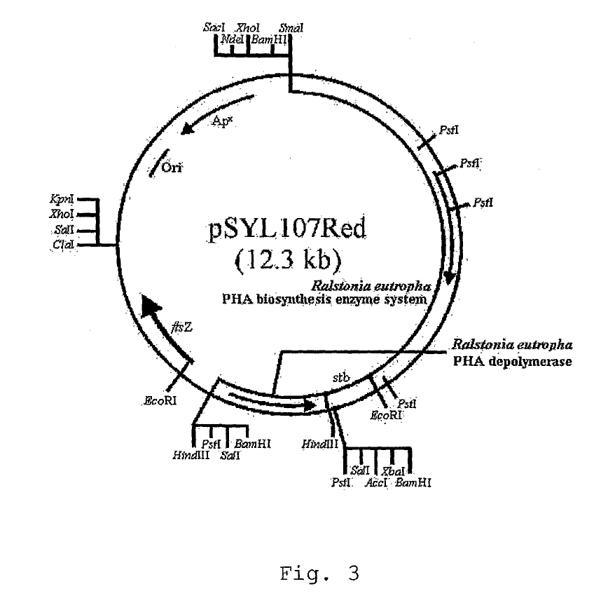

FIGS. 1, 2 and 3 are genetic maps of the said constructed plasmids, pJC4Red, pSYL105Red and pSYL107Red, respectively. The DNA fragments inserted into the said plasmids, pJC4Red, pSYL105Red and pSYL107Red contain the constitutive promoter of a gene for intracellular PHA polymerase of Ralstonia eutropha.

Among three recombinant E. coli above, E. coli XL1-Blue transformed with recombinant plasmids, pJC4Red and pSYL105Red, respectively, were named E. coli XL1-Blue/pJC4Red (Escherichia coli XL1-Blue/pJC4Red) and E. coli XL1-Blue/pSYL105Red (Escherichia coli XL1-Blue/pSYL105Red), which were deposited with the Korean Collection for Type Cultures (KCTC, #52, Oun-dong, Yusong-ku, Taejon 305-333, Republic of Korea) affiliated to Korea Research Institute of Bioscience and Biotechnology (KRIBB), an international depository authority, under Accession No. KCTC 0677BP and KCTC 0676BP on Oct. 22, 1999, respectively.

### EXAMPLE 2

### Preparation of (R)-3-hydroxybutyrate

The plasmids, pJC4Red, pSYL105Red and pSYL107Red constructed in Example 1 were introduced into E. coli XL1-Blue by electroporation technique to obtain three kinds of recombinant E. coli, i.e., E. coli XL1-Blue/pJC4Red, E. coli XL1-Blue/pSYL105Red and E. coli XL1-Blue/pSYL107Red. The three types of recombinant E. coli thus obtained were cultured respectively in a LB medium containing 100 mg/L ampicillin for 12 hours and then 1 ml aliquot of each culture broth was inoculated into 100 ml R medium (see: Lee and Chang, Biotechnol. Lett., 15:971-974, 1993) containing 20 g/L glucose and 20 mg/L thiamine in a 250 ml flask, respectively. Recombinant E. coli XL1-Blue/pSYL107Red was cultured at 30° C. and E. coli XL1-Blue/pSYL105Red and E. coli XL1-Blue/pJC4Red were cultured at 37° C. under a rotary shaking condition of 250 rpm, respectively. And then, cell concentration in dried mass, PHB concentration, PHB content, monomer ((R)-3-hydroxybutyrate) concentration and dimer concentration were measured, whose results are shown in Table 1. Dimers were found in the medium, since dimers were easily exported into the medium following digestion of ester bond of accumulated intracellular PHB by depolymerase of Ralstonia eutropha.

PHB content in Table 1 was defined as weight of accumulated PHB per unit mass of dried cell, and the final yield was defined as sum of monomer concentration and dimer concentration which was converted into monomer concentration per unit mass of glucose. Although the concentrations of (R)-3-hydroxybutyrate monomer were as low as 1.6 g/L for recombinant E. coli XL1-Blue/pSYL105Red, 1.7 g/L for recombinant E. coli XL1-Blue/pSYL107Red, and 0.7 g/L for recombinant E. coli XL1-Blue/pJC4Red, concentrations of dimer were as high as 6.1, 2.7 and 6.7 g/L, respectively, which can be converted into monomer by heating under a basic condition, giving 44, 25 and 43% final yields of (R)-3-hydroxybutyrate for the substrate glucose, respectively.

TABLE 1
Preparation of (R)-3-hydroxybutyrate

| | | | Conc. | | Monomer | Dimer | |
|---|---|---|---|---|---|---|---|
| | Culture | Conc. of | of | PHB | concen- | concen- | Final |
| Plasmid/ | temp./ | dried | PHB | content | tration | tration | yield |
| Media | Time | cell (g/L) | (g/L) | (%) | (g/L) | (g/L) | (%) |
| pJC4Red/ | 37° C./ | 1.45 | 0.10 | 7 | 0.7 | 6.7 | 43 |
| R + glucose + | 51 hrs | | | | | | |
| thiamine | | | | | | | |
| pSYL105Red/ | 37° C./ | 1.50 | 0.47 | 31 | 1.6 | 6.1 | 44 |
| R + glucose + | 51 hrs | | | | | | |
| thiamine | | | | | | | |
| pSYL107Red/ | 30° C./ | 3.04 | 1.97 | 65 | 1.7 | 2.7 | 25 |
| R + glucose + | 51 hrs | | | | | | |
| thiamine | | | | | | | |
| pJC4Red/ | 37° C./ | 2.72 | 1.20 | 44 | 0.2 | 2.6 | 16 |
| LB + glucose | 51 hrs | | | | | | |
| pSYL105Red/ | 37° C./ | 2.75 | 1.32 | 48 | 0.2 | 4.0 | 25 |
| LB + glucose | 51 hrs | | | | | | |
| pSYL107Red/ | 30° C./ | 3.84 | 2.81 | 73 | 0.9 | 0.3 | 6 |
| LB + glucose | 51 hrs | | | | | | |

In order to show the expression of above plasmids in other species of E. coli, 12 types of recombinant E. coli were prepared by transforming above 3 plasmids into E. coli B (ATCC 11303), HB101 (see: Boyer and Roulland-Dussoix, J. Mol. Biol., 41:459-472, 1969), JM101 (see: Messing et al. Nucleic Acids Res., 9:309-321, 1981) and W3110 (ATCC 27325) employing electroporation technique, respectively. Each recombinant E. coli was cultured in a LB medium containing 100 mg/L ampicillin for 12 hours and then 1 ml aliquot of each culture broth was inoculated into 100 ml LB medium containing 20 g/L glucose in a 250 ml flask, respectively. After 51 hour incubation, approximately 0.1 to 0.3 g/L (R)-3-hydroxybutyrate monomer and approximately 2 g/L dimer were secreted into the medium, which are relatively lower than the yields with the E. coli XL1-Blue. Therefore, it was clearly demonstrated that the plasmid system constructed above can be employed to various E. coli strains, and other various E. coli strains than 4 types of E. coli strain employed in the present Example may be used.

The PHA depolymerase gene of Ralstonia eutropha in the plasmids used in the Example was expressed from the intrinsic constitutive promoter of Ralstonia eutropha, however, it will be understood by the conventionally skilled in the art that similar results can be obtained by substituting the said promoter with other constitutive promoters which act in other strains of E. coli.

Also, plasmids used in the present Example contain a gene coding for intracellular PHA depolymerase of Ralstonia eutropha and a gene coding for PHA biosynthesis enzyme of Alcaligenes latus or Ralstonia eutropha, in cis. However, it will be understood by the conventionally skilled in the art that similar results can be obtained by cotransforming E. coli with the recombinant plasmids containing different but compatible origin of replication (i.e., pBR322 or pUC19-derived plasmids carrying ColE1 compatible origin of replication, or pACY177 or pACYC184-derived plasmids carrying p15A origin of replication) into which these genes are cloned separately, in trans.

### EXAMPLE 3

### Construction of Plasmid Vector System

Since the plasmids constructed in Example 1 express PHA depolymerase from the intrinsic constitutive promoter of Ralstonia eutropha, synthesis and degradation occur simultaneously. Thus, to find out the possibility of using an inducible promoter instead of constitutive promoter, experiments were conducted as follows. In order to control time of PHA depolymerase expression and to get high level of expression, a strong inducible trc promoter (see: Amann and Brosius, Gene, 40:183-190, 1985) was introduced to construct plasmids containing inducible PHA depolymerase gene of Ralstonia eutropha. Of course, it will be understood by the conventionally skilled in the art that, besides trc promoter, E. coli inducible promoters such as T7 promoter (see: Caton and Robertson, Nucleic Acids Res., 7:1445-1456, 1979), trp promoter (see: Yanofsky et al., Nucleic Acids Res., 9:6647, 1981), tac promoter (see: de Boer, Proc. Natl. Acad. Sci., USA, 80:21-25, 1983), and bad promoter (see: Smith and Schleif, J. Biol. Chem., 253:6931-6933, 1978) may be used.

First, PCR was performed using Ralstonia eutropha DNA isolated as in Example 1 as a template, primer 3, 5′-GCTACGTAGGTCTCGCATGCTCTACCAATTGCATG-3′ (SEQ ID NO: 3), primer 4, 5′-CGGGATCCAAGCTTACCTGGTGGCCGAGGC-3′ (SEQ ID NO: 4) and DNA polymerase under the same condition described in Example 1. DNA obtained from the PCR was subjected to agarose gel electrophoresis to isolate approximately 1.4 kbp DNA fragment which was subsequently double digested with BsaI and HindIII. Separately, the plasmid pTrc99A containing strong inducible promoter was double digested with NcoI and HindIII. DNA fragment obtained above was cloned into the digested plasmid pTrc99A to construct a recombinant plasmid pTrc99ARed. pTrc99ARed was introduced into E. coli XL1-Blue employing electroporation technique and then transformed E. coli were selected on LB agar plate containing 50 mg/L ampicillin to obtain a recombinant E. coli XL1-Blue/pTrc99ARed. The said recombinant E. coli was cultured in LB liquid medium containing 100 mg/L ampicillin and then DNA of recombinant plasmid pTrc99ARed was prepared in a large scale by alkaline lysis technique.

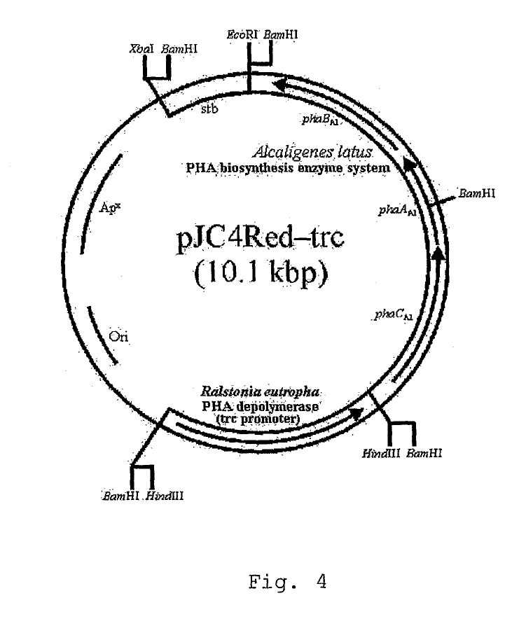

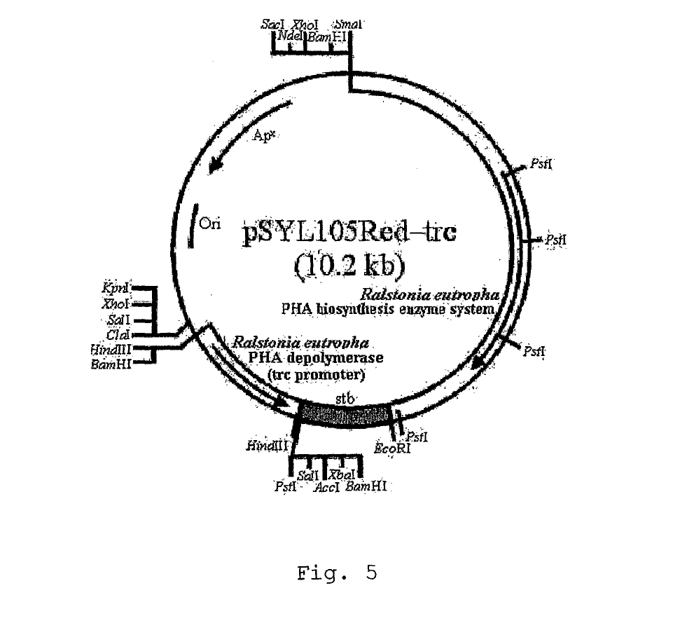

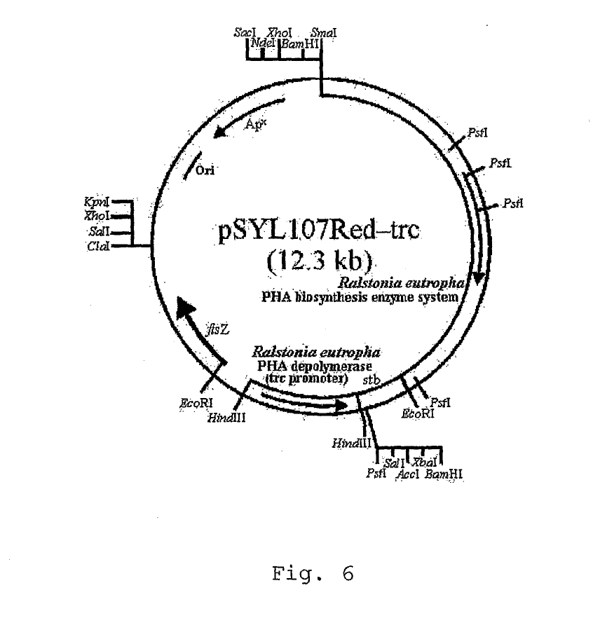

To obtain DNA fragments containing intracellular PHA depolymerase gene carrying inducible trc promoter, PCR was performed using the recombinant plasmid pTrc99ARed constructed above as a template, primer 5, 5′-GCAAGCTTCGACTGCACGGTGCACC-3′ (SEQ ID NO: 5), primer 6, 5′-CGGGATCCAAGCTTACCTGGTGGCCGAGGC-3′ (SEQ ID NO: 6) and DNA polymerase under the same condition described in Example 1. DNAs obtained from the PCR was subjected to agarose gel electrophoresis to isolate approximately 1.6 kbp DNA fragment which was subsequently digested with HindIII and cloned into the HindIII site of plasmid pJC4 containing a gene for PHA biosynthesis enzyme of Alcaligenes latus and two kinds of plasmid pSYL105 and pSYL107 both containing a gene for PHA biosynthesis enzyme of Ralstonia eutropha, respectively, to obtain recombinant plasmids pJC4Red-trc, pSYL105Red-trc and pSYL107Red-trc. FIGS. 4, 5 and 6 are genetic maps of the plasmids pJC4Red-trc, pSYL105Red-trc and pSYL107Red-trc constructed above, respectively. Among three recombinant E. coli above, E. coli XL1-Blue transformed with recombinant plasmid, pSYL105Red-trc was named E. coli XL1-Blue/pSYL105Red-trc (Escherichia coli XL1-Blue/pSYL105Red-trc), which was deposited with the Korean Collection for Type Cultures (KCTC, #52, Oun-dong, Yusong-ku, Taejon 305-333, Republic of Korea) affiliated to Korea Research Institute of Bioscience and Biotechnology (KRIBB), an international depository authority, under Accession No. KCTC 0678BP on Oct. 22, 1999.

### EXAMPLE 4

### Preparation of (R)-3-hydroxybutyrate Employing trc Promoter

The plasmids, pJC4Red-trc, pSYL105Red-trc and pSYL107Red-trc constructed in Example 3 were introduced into E. coli XL1-Blue by electroporation technique to obtain three kinds of recombinant E. coli, i.e., E. coli XL1-Blue/pJC4Red-trc, E. coli XL1-Blue/pSYL105Red-trc and E. coli XL1-Blue/pSYL107Red-trc. Recombinant E. coli obtained above were cultured in R medium containing 20 g/L glucose and 20 mg/L thiamine in a 250 ml flask, respectively. Recombinant E. coli XL1-Blue/pSYL105Red-trc and E. coli XL1-Blue/pJC4Red-trc were cultured at 37° C. and E. coli XL1-Blue/pSYL107Red-trc was cultured at 30° C. under a rotary shaking condition of 250 rpm for 2 days, to which 1 mM IPTG was added to induce the expression of intracellular PHA depolymerase, and the production of (R)-3-hydroxybutyrate was measured with time. Cell concentration in dried mass, PHB concentration, PHB content, monomer ((R)-3-hydroxybutyrate) concentration and dimer concentration were measured and the results are shown in Table 2.

TABLE 2
Preparation of (R)-3-hydroxybutyrate employing
trc promoter

| | Culture | Conc. of | | | Monomer | Dimer | |
|---|---|---|---|---|---|---|---|
| Plasmid/ | time | dried | Conc. | PHB | concen- | concen- | Final |
| Culture | after | cell | of PHB | content | tration | tration | yield |
| temp. | induction | (g/L) | (g/L) | (%) | (g/L) | (g/L) | (%) |
| pJC4Red- | 2 hrs | 1.4 | 0.16 | 11 | 0.1 | 3.6 | 22 |
| trc/ | 4 hrs | 1.2 | 0.09 | 8 | 0.5 | 4.3 | 28 |
| 37° C. | 6 hrs | 1.1 | 0.09 | 8 | 0.5 | 5.0 | 32 |
| pSYL105Red- | 2 hrs | 1.2 | 0.04 | 3 | 0.7 | 6.0 | 39 |
| trc/ | 4 hrs | 1.3 | 0.00 | 0 | 0.7 | 6.1 | 40 |
| 37° C. | 6 hrs | 1.3 | 0.02 | 2 | 0.7 | 7.1 | 45 |
| pSYL107Red- | 2 hrs | 0.7 | 0.12 | 17 | 0.0 | 0.6 | 4 |
| trc/ | 4 hrs | 0.8 | 0.15 | 19 | 0.1 | 1.2 | 8 |
| 30° C. | 6 hrs | 0.8 | 0.15 | 19 | 0.3 | 1.5 | 10 |

PHB content in Table 2 was defined as weight of accumulated PHB per unit mass of dried cell, and the final yield was defined as sum of monomer concentration and dimer concentration which was converted into monomer concentration per unit mass of glucose.

As shown above, it was possible to prepare optically pure (R)-3-hydroxybutyrate efficiently by expressing intracellular PHA depolymerase of Ralstonia eutropha employing inducible promoter. Thus, it may be possible to obtain optically pure (R)-3-hydroxybutyrate by expressing Alcaligenes latus-derived intracellular PHA depolymerase from the inducible promoter.

### EXAMPLE 5

### Preparation of (R)-3-hydroxybutyrate and (R)-3-Hydroxyvalerate in a Simultaneous Manner

To examine whether other hydroxycarboxylic acids than (R)-3-hydroxybutyrate can be prepared employing recombinant E. coli, 4 kinds of recombinant E. coli transformed with the above-constructed 4 recombinant plasmids, pJC4Red, pSYL107Red, pJC4Red-trc and pSYL107Red-trc containing intracellular PHA depolymerase of Ralstonia eutropha were cultured in R medium containing 10 g/L glucose, 1 g/L propionic acid and 20 mg/L thiamine, respectively. Culture temperature for the recombinant E. coli transformed with pSYL107Red or pSYL107Red-trc which carries PHA biosynthesis enzyme of Ralstonia eutropha was 30° C. and culture temperature for the recombinant E. coli transformed with pJC4Red or pJC4Red-trc which carries PHA biosynthesis enzyme of Alcaligenes latus was 37° C., and culture was continued for 48 hours under a rotary shaking condition of 250 rpm. Among them, recombinant E. coli transformed with plasmids carrying trc promoter were cultured for 48 hours, induced the expression of the enzyme by adding 1 mM IPTG (β-isopropylthiogalactoside), cultured for 4 hours, and then, cell concentration in dried mass, PHB concentration, PHB content, concentrations of monomers, (R)-3-hydroxybutyrate and (R)-3-hydroxyvalerate, were measured, respectively, whose results are shown in Table 3.

TABLE 3
Preparation of (R)-3-hydroxybutyrate and (R)-3-
hydroxyvalerate in a simultaneous manner

| | Culture | Conc. | | Conc. of |
|---|---|---|---|---|
| | temp./Cul- | of | Conc. of | (R)-3- |
| | ture time | dried | (R)-3- | hydroxy- |
| Recombinant | after | cell | hydroxy- | valerate |
| E. coli | induction | (g/L) | butyrate (g/L) | (g/L) |
| XL1-Blue/pJC4Red | 37° C./0 hr | 2.15 | 0.12 | 0.01 |
| XL1- | 30° C./0 hr | 1.10 | 0.10 | 0.00 |
| Blue/pSYL107Red | | | | |
| XL1-Blue/pJC4Red- | 37° C./4 hrs | 0.80 | 1.51 | 0.28 |
| trc | | | | |
| XL1- | 37° C./4 hrs | 0.95 | 0.17 | 0.03 |
| Blue/pSYL107Red- | | | | |
| trc | | | | |

As shown in Table 3, it was clearly demonstrated that (R)-3-hydroxybutyrate and (R)-3-hydroxyvalerate can be produced efficiently in a simultaneous manner employing recombinant E. coli. Similarly, various hydrocarboxylic acids may be produced by degrading other PHAs which can be synthesized by recombinant E. coli, furthermore, various monomers of PHA may be prepared by controlling culture conditions, microorganism strains, PHA synthesis/degradation system and their combinations (see: Steinbuchel and Valentin, FEMS Microbiol. Lett., 128:219-228, 1995; Lee et al., Biotechnol. Bioeng., 65:363-368, 1999).

As clearly illustrated and demonstrated above, the present invention provides a process for preparing (R)-3-hydroxycarboxylic acid by culturing E. coli transformed with a plasmid containing a gene for PHA biosynthesis enzyme and a gene for PHA depolymerase in cis. In accordance with the present invention, (R)-3-hydroxycarboxylic acids such as (R)-3-hydroxybutyrate and (R)-3-hydroxyvalerate can be secreted directly into the medium by simple culture of recombinant E. coli containing PHA biosynthesis enzyme system and PHA depolymerase system, and subsequent induction of PHA depolymerase, which simplifies the whole process into just two steps of culture and isolation. Furthermore, continuous process can be employed, and when cells are immobilized, disposal of cell waste can be avoided or reduced significantly, to increase the total yield of product. Also, the said recombinant E. coli system can be used widely in preparing various 3-hycroxycaboxylic acids by cloning of a gene for PHA biosynthesis enzyme and a gene for PHA depolymerase which can produce PHAs including other monomer(s) than (R)-3-hydroxybutyrate or (R)-3-hydroxyvalerate.

[image]

[image]

[image]

What is claimed is:

1. A recombinant plasmid comprising a nucleotide sequence encoding a polyhydroxyalkanoate (PHA) biosynthesis enzyme and a nucleotide encoding a PHA depolymerase.

2. The recombinant plasmid of claim 1, wherein the nucleotide encoding the PHA biosynthesis enzyme is derived from Alcaligene latus or Ralstonia eutropha.

3. The recombinant plasmid of claim 1, wherein the nucleotide encoding the PHA depolymerase is derived from Ralstonia eutropha.

4. The recombinant plasmid of claim 1, wherein the nucleotide encoding the PHA depolymerase employs a promoter selected from the group consisting of intrinsic constitutive promoter of Ralstonia eutropha, trc, T7, trp, tac and bad inducible promoter.

5. The recombinant plasmid of claim 1, wherein the plasmid is selected from the group consisting of pJC4Red, pSYLI05Red, pSYLI07Red, pJC4Red-trc, pSYLI05Red-trc and pSYLI07Red-trc.

6. A bacterial cell comprising a recombinant plasmid, wherein the bacterial cell is Escherichia coli XLI-Blue, and wherein the recombinant plasmid is pJC4Red.

7. The bacterial cell of claim 6, wherein the cell is Escherichia coli XLI-Blue/pJC4Red, deposited with deposit No. KCTC 0677BP.

8. A bacterial cell comprising a recombinant plasmid, wherein the bacterial cell is Escherichia coli XLI-Blue, and wherein the recombinant plasmid is pSYLI05Red

9. The bacterial cell of claim 8, wherein the cell is Escherichia coli XLI-Blue/pSYL105Red, deposited with deposit No. KCTC 0676BP.

10. A bacterial cell comprising a recombinant plasmid, wherein the bacterial cell is Escherichia coli XLI-Blue, and wherein the recombinant plasmid is pSYL105Red-trc.

11. The bacterial cell of claim 10, wherein the cell is Escherichia coli XLI-Blue/pSYLI05Red-trc, deposited with deposit No. KCTC 0678BP.

12. A method of preparing a hydroxycarboxylic acid, comprising:
culturing a population of bacterial cells transformed with the recombinant plasmid of claim 1 in a culture medium adapted to produce a hydroxycarboxylic acid; and
isolating the hydroxycarboxylic acid from the culture medium.

13. The method of claim 12, wherein the recombinant the plasmid is selected from group consisting of pSYLI05Red, pSYLI07Red, pJC4Red-trc, pSYLI05Red-trc and pSYLI07Red-trc.

14. The method of claim 12, wherein the population of bacterial cells comprises one or more selected from the group consisting of Escherichia coli XLI-Blue/pJC4Red (KCTC 0677BP), Escherichia coli XL1-Blue/pSYL105Red (KCTC 0676BP), and Escherichia coli XLI-Blue/pSYLI05Red-trc (KCTC 0678BP).

15. The method of claim 12, wherein the hydroxycarboxylic acid is (R)-3-hydroxycarboxylic acid.

16. The method of claim 12, wherein the hydroxycarboxylic acid is selected from the group consisting of (R)-3-hydroxybutyrate, (R)-3-hydroxyvalerate, dimer of (R)-3-hydroxybutyrate, dimer of (R)-3-hydroxyvalerate, an ester of (R)-3-hydroxybutyrate, an ester of (R)-3-hydroxyvalerate or a mixture thereof.
